(19) United States
(12) Patent Application Publication
(10) Pub. No.: US 2012/0087907 A1
(43) Pub. Date: Apr. 12, 2012

(54) PROTHROMBIC COMPLEX COMPOSITION

(75) Inventor: Jacques Chabbat, PARIS (FR)

(73) Assignee: LABORATOIRE FRANCAIS DU FRACTIONNEMENT ET DES BIOTECHNOLOGIES SA, LES ULIS (FR)

(21) Appl. No.: 13/376,316

(22) PCT Filed: Jun. 4, 2010
(86) PCT No.: PCT/IB10/52497

§ 371 Date: Dec. 5, 2011

(30) Foreign Application Priority Data
Jun. 5, 2009 (FR) 09/02723

(51) Int. Cl. A61K 38/48; A61P 7/04; C12N 9/74

(52) U.S. Cl. 424/94.64; 435/214

(57) ABSTRACT

The present disclosure relates to a method for preparing a composition or a concentrate of a prothrombic complex that includes the II, VII, IX and X coagulation factors, including providing a supernatant of a plasma cryoprecipitate, applying the supernatant on an anion-exchange resin for producing an eluate containing the complex and proteins having a high molecular weight, and applying the eluate on a hydroxyapatite column for producing a second eluate containing the complex. The disclosure also relates to a composition that can be produced by the method.

### CROSS-REFERENCE TO RELATED APPLICATIONS

This application is a National Phase Entry of International Application No. PCT/IB2010/052497, filed on Jun. 4, 2010, which claims priority to French Patent Application Serial No. 0902723, filed on Jun. 5, 2009, both of which are incorporated by reference herein.

### FIELD OF THE INVENTION

The present invention relates to a method for preparing a composition or a concentrate of a prothrombic complex comprising the coagulation factors II, VII, IX and X. The invention further provides a composition obtainable by this method.

### BACKGROUND

Making concentrates of proteins depending on vitamin K and, more particularly concentrates of prothrombic complex comprising the coagulation factors II, VII, IX and X (also called PPSB) is primordial for preventing or treating hemorrhagic accidents in hemophilic patients who suffer from a deficiency in one or more of the coagulation factors and/or who suffer from a deficiency for certain proteins with high molecular weights. During a hemorrhagic phase, the patients are subject to a treatment with PPSB and therefore receive large doses of plasma proteins usually co-purified with PPSB, which may cause the occurrence of secondary effects such as anaphylactic shocks, reactions of the inflammatory type and problems of tolerance. This is notably the case with immunoglobulins M and the factors C3a, C4a or C5a, also called anaphylatoxins which result from the activation of the factors C3, C4, C5. The factors C3a, C4a and C5a (particularly C3a) are involved in allergic inflammation. Activation of the mastocytes and basophils by the fragments C5a and C3a of the complement actually causes release of leukotrienes and of histamine, at the origin of an increase in the capillary permeability, in bronchoconstriction and in vasodilation. In order to avoid the formation of these fragments during the method for purifying the vitamin-K-dependent factors, it is therefore preferable to remove their respective precursors, i.e. the factors C3, C4 or C5 of the system of the complement.

The PPSB concentrates commercially available to this day, such as Kaskadil® (from the “Laboratoire Français du Fractionnement et des Biotechnologies”) all comprise a large proportion (about 80%) of contaminating proteins other than the factors II, VII, IX and X making up the PPSB. The vitamin-K-dependent protein for which the concentration is the largest is prothrombin or factor II (with a concentration of the order of 4.5 Mg/mL for a concentration of total proteins in the Kaskadil of about 35-45 Mg/mL). Therefore, there exists a significant need for a method allowing a higher degree of purification of PPSB, and capable of preserving the activity and the respective proportion of the factors II, VII, IX and X which make it up.

Document EP-A-0528701 (“Association pour l'essor de la transfusion sanguine”) describes a method for preparing human thrombin intended for therapeutic use and comprising successive steps for purifying a plasma cryoprecipitate supernatant on a DEAE-Sephadex® A50 resin, for recalcifying and virally inactivating the eluate containing the PPSB. U.S. Pat. No. 4,411,794 describes a method for purifying the coagulation factors II, VII, IX and X comprising a step for adsorption of a plasma precipitation supernatant with ammonium sulfate on a mineral support of the hydroxyapatite type in the presence of calcium ions, followed by a purification step on colloidal silica and by dialysis. However it appears that the PPSB concentrate which results from this comprises many contaminating proteins and does not have the required degree of purity for meeting present criteria as regards health safety of blood-derived products. Ammonium sulfate in particular is not suitable for therapeutic use and has relative toxicity.

U.S. Pat. No. 4,272,523 describes a method for fractionation of plasma from a plasma cryoprecipitate supernatant. This patent notably describes the preparation of a PPSB concentrate by accumulating the adsorption steps of the cryoprecipitate supernatant on colloidal silica, for dialysis/ultrafiltration, adsorption on tricalcium phosphate of the hydroxyapatite type, and adsorption on an anion exchange resin of the DEAE-Sephadex type. However, it appears that the purification step on colloidal silica used for removing the high molecular weight proteins such as fibrinogen and the purification step on tricalcium phosphate take place in the form of batchwise adsorption (in batches), an implementation which, by its low reproducibility and its difficulty of being automated, proves to be difficult to apply on an industrial scale. Indeed, tricalcium phosphate is difficult to control since it appears as a powder sensitive to hygrometry and including intrinsic characteristics which may vary depending on the batches. The method of U.S. Pat. No. 4,272,523 therefore proves to be unsuitable for large scale preparation of PPSB concentrates intended for therapeutic use.

Document EP-A-0832200 describes a method for purifying a composition of recombinant FXI, comprising the successive chromatography steps on anion exchange resin, on heparin resin and then on hydroxyapatite resin. This document does not relate to a factor of the prothrombic complex and the starting product is a composition of a recombinant factor and not of human origin. Document WO2006/074664 describes a method for purifying recombinant FVII comprising a chromatography step on hydroxyapatite, without prior treatment of the composition containing the recombinant FVII.

### SUMMARY

The applicant surprisingly found that a method for purifying a concentrate of proteins depending on vitamin K, notably a prothrombic complex, combining the steps for preparing a supernatant of plasma cryoprecipitate, a chromatography on anion exchange resin and a chromatography on hydroxyapatite, allows industrial preparation of a PPSB concentrate having a high degree of purity. The PPSB prepared by the invention is substantially without any contaminating proteins and the factors II, VII, IX and X which it contains, have high specific activity. The method of the invention is most particularly distinguished from the purification methods known to this day by the reduced number and the reproducibility of the implemented purification steps, and by the use of hydroxyapatite to chromatograph an eluate containing proteins with a high molecular weight such as fibrinogen, fibronectin, immunoglobulins, proteins of the complement.

The object of the present invention is a method for preparing a prothrombic complex composition comprising the following steps:

a) providing a supernatant of a plasma cryoprecipitate,

b) applying said supernatant on an anion exchange resin, and eluting in an eluate containing said complex and proteins of high molecular weight,

c) applying the eluate resulting from step b) on a hydroxyapatite column,

d) eluting in an eluent containing said complex.

In a preferred embodiment, the method of the invention comprises an additional pre-elution step c1), said pre-elution being preferably carried out at a Ph comprised between 6.5 and 8.5, preferably about 8, with a sodium phosphate or potassium phosphate buffer, notably in a concentration from 0.005 to 0.05 M, advantageously from 0.01 to 0.05 M, advantageously from 0.02 to 0.05 M, and preferably 0.03 M, said buffer also comprising 0.25 M NaCl. More preferably, the elution of step d) is carried out with a potassium phosphate buffer of preferably 0.5 M, 0.075 M NaCl, Ph 8.

Advantageously, the method of the invention comprises at least one additional step for viral inactivation of the eluate resulting from step b) and/or of the eluate resulting from step d). In a preferred embodiment, said at least one viral inactivation step is carried out on the eluate resulting from step b) in the form of a solvent-detergent treatment, preferably in the presence of a Tween (polysorbate 80)—TnBP mixture, preferably with a 1% (v/v) polysorbate 80—0.3% (v/v) TnBP mixture. In a particular embodiment, said at least one viral inactivation step is carried out as a UV-C (Ultra Violet C) treatment, treatment with caprylate ions and/or by dry heating. Advantageously, said at least one viral inactivation step is completed by a viral removal step carried out on the eluate resulting from step d) as a nanofiltration, for example one or several nanofiltrations on one or several filters with a porosity for example comprised between 15 nm and 100 nm, preferably on at least one filter with a porosity of for example 15 nm, notably on a Planova 15N filter from Asahi.

In a particular embodiment, the method of the invention comprises at least one additional diafiltration-ultrafiltration step after step b) and/or after step d). In a particular embodiment, step b) of the method of the invention comprises two sub-steps implemented on two distinct anion exchange resins. In a particular embodiment, the anion exchange resin of step b) has a positively charged group selected from diethylaminoethane (DEAE), polyethylene-imine (PEI) and quaternary aminoethane (QAE), said anion exchange resin preferably being of the DEAE type.

In a particular embodiment, the method of the invention comprises the addition of an inhibitor of thrombin, preferably anti-thrombin III or a mixture of anti-thrombin III and of heparin after step b) or after step d). In an embodiment, the composition prepared by the method of the invention further comprises other proteins depending on vitamin K such as the proteins C, S and Z. In a particular embodiment, the method according to the invention comprises a final additional formulation step, preferably by freeze-drying and/or addition of pharmaceutically acceptable adjuvants or carriers.

The present invention also relates to a composition of prothrombic complex obtainable by the method according to the invention, and for which the immunoglobulin concentration, and preferably the IgM concentration, is less than 0.1%, and/or for which the fibrinogen concentration is less than 0.1%, and/or for which the fibronectin concentration is less than 0.1% and/or for which the concentration of factors of the complement is less than 0.1%. In a particular embodiment, the average specific activity of the FIX in the composition of prothrombic complex according to the invention is of at least 4 IU per mg of proteins. In a particular embodiment, the composition of prothrombic complex according to the invention further comprises the protein C, protein S and protein Z.

In a particular embodiment, the proteins depending on vitamin K, constituted by the Factor II (FII), the Factor VII (FVII), the Factor IX (FIX), the Factor X (FX), protein C, protein S and protein Z of the prothrombic complex composition according to the invention account for a minimum of 80%, preferably 85%, and more preferably 90%, of the total proteins of the composition. The present invention also relates to the use of the prothrombic complex composition according to the invention as a drug, preferably as a drug for treating and preventing hemorrhagic accidents related to deficiency in factors dependent on vitamin K, or to overdosage of antivitamin K, or as a drug for treating and preventing hemorrhagic accidents related to a constitutional or acquired deficiency in factor II or in factor X.

### BRIEF DESCRIPTION OF THE DRAWINGS

FIG. 1: Elution yields in factors II, IX, VII and X versus the protein load deposited on the hydroxyapatite column.

FIG. 2: Amount of factors II, IX, VII and X not retained on hydroxyapatite column versus the introduced protein load.

FIG. 3: Percentage variation of non-bound Ell and FIX versus the protein load deposited on a hydroxyapatite column.

FIG. 4: SDS-PAGE gel corresponding to the amounts of proteins removed during pre-elution during the chromatography on hydroxyapatite. 12% SDS-PAGE gel—non-reduced—deposits: 10 μg of proteins, corresponding to pre-eluates and eluates from chromatography on HA Biorad. Wells 1 and 10: Molecular weight standards. Well 2: PI-1. Well 3: Test 3 pre-eluate 0.25 M NaCl. Well 4: Test 3 eluate. Well 5: Test 5 pre-eluate 0.25 M NaCl; 30 mM phosphate. Well 6: Test 5 eluate. Well 7: PI-1. Well 8: Test 6 pre-eluate 0.25 M NaCl; 30 mM phosphate. Well 9: Test 6 eluate.

FIG. 5: Change in the filtration pressure versus time.

FIG. 6: Change in the filtration flow rate versus the filtered weight.

FIG. 7: Electrophoresis on 4—12% SDS-PAGE Novex without any reducing agent—colloidal Coomassie blue staining. Well 1: 97 E 0801—PI-1. Well 2: 97 E 0801—non-adsorbed HA ceramic. Well 3: 97 E 0801 —pre-elution. Well 4: 97 E 0801—elution. Well 5: 97 E 0801—dialyzed elution 10 kDa. Well 6: 97 E 0901—after 15 nm filtration. Well 7: 97 E 1401—after 15 nm filtration. Well 8: 97 E 1601—after 15 nm filtration. Well 9: 97 E 1601—final retentate 15 nm. Well 10: Novex molecular weight control.

FIG. 8: Characterization of the factor IX by immunoblot. Well 1: 97 E 1106—dialyzed HA eluate before nanofiltration. Wells 2 and 3: 97 E 1106—PI-1. Well 4: 97 E 1504—15 nm filtrate. Well 5: Factor IXHP control. The Factor IXHP is a Factor IX of high purity, i.e. a Factor IX concentrate having a specific activity (expressed in units of FIX per mg of proteins) of greater than 100 U/mg.

### DETAILED DESCRIPTION OF EMBODIMENTS

The method for purifying proteins dependent on vitamin K and notably a prothrombic complex of the present invention comprises the following steps:

a) providing a supernatant of a plasma cryoprecipitate. In a particular embodiment, the plasma cryoprecipitate supernatant may be obtained by Cohn fractionation. In this particular case, it proves to be necessary to avoid denaturation of the proteins by ethanol and therefore to operate at a low temperature or remove the alcohol before proceeding with the adsorption of the proteins on hydroxyapatite. In another embodiment, the plasma cryoprecipitate supernatant may be obtained by fractionation with an ammonium sulfate salt. In this particular case, it proves to be necessary to perform dialysis in order to be under optimum conditions for adsorption on hydroxyapatite.

b) applying said supernatant on an anion exchange resin, and eluting in an eluate containing said complex and proteins of high molecular weight,

c) applying the eluate resulting from step b) on a hydroxyapatite column,

d) eluting in an eluent containing said complex.

The proteins of high molecular weights are those having a MW expressed in kDa of more than 300, preferably more than 200, notably more than 160, or even more than 100. The hydroxyapatite resin used in the invention may for example be ceramic-hydroxyapatite (ceramic HA), Biogel HT, etc.

In a preferred embodiment, the method of the invention comprises an additional pre-elution step c1), said pre-elution being preferably carried out at room temperature with a 0.01 M potassium phosphate buffer, 0.25 M NaCl, pH 8.0 or a 0.03 M potassium phosphate buffer, 0.25 M NaCl, pH 8.0. The potassium phosphate concentration of the pre-elution buffer preferably varies from 0.02 to 0.05 M, and is preferentially equal to 0.03 M. The pH of the pre-elution buffer preferably varies from pH 6.5 to pH 8.5, and is preferentially equal to pH 8.

Preferentially, the elution of step d) is carried out with a 0.5 M potassium phosphate buffer, 0.075 M NaCl, pH 8. The pH of the elution buffer preferably varies from pH 6.5 to pH 8.5, and is preferentially equal to pH 8. The potassium phosphate concentration of the elution buffer preferably varies from 0.1 M to 0.5 M and is preferentially equal to 0.25 M.

With the chromatography on hydroxyapatite column and, preferably on ceramic hydroxyapatite (HA-Biorad), it is possible to remove the proteins of high molecular weight, which are eluted with the proteins depending on vitamin K (notably the prothrombic complex) during the chromatography step b) on anion exchange resin. By removing these proteins of high molecular weight it is possible to reduce or preferably remove the secondary effects which generally result from the therapeutic use of a prothrombic complex solution. Indeed, the contaminating proteins of high molecular weight which are removed during chromatography on hydroxyapatite for example comprise certain factors of the complement (such as C4) which reduce either directly or indirectly (for example after cleavage in the form of anaphylatoxins) the tolerance of the patient to solutions of prothrombic complex commercially distributed today. The applicant surprisingly found that with the single chromatography step on hydroxyapatite, it is possible to remove most of the contaminating proteins of high molecular weight contained in the plasma cryoprecipitate supernatant without requiring a preliminary implementation such as purification on colloidal silica, for example. Proteins such as fibrinogen, fibronectin, Ig are also removed.

Chromatography on hydroxyapatite further gives the possibility of not changing the respective proportion of the factors dependent on vitamin K during their purification, since the ratio between the factors II, VII, IX or X in the prothrombic complex concentrate obtained by the method of the invention is extremely comparable to the one encountered in native plasma. The result is that the prothrombic complex composition (concentrate of proteins dependent on vitamin K) resulting from chromatography on hydroxyapatite is enriched significantly. The content, relatively to the protein content, of this composition or concentrate of proteins of the prothrombic complex is of about 60%, preferably about 70%, and more preferably about 80%, and the specific activity of the factors II, VII, IX, X is significantly increased relatively to the commercially distributed prothrombic concentrates (from 4 to 8 times greater, preferably 5 times greater), like for example Kaskadil®.

Moreover, removal of the proteins of high molecular weight during chromatography on hydroxyapatite is an industrial advantage in that from now on, it allows viral inactivation of the concentrate of proteins dependent on vitamin K by nanofiltration on filters with a porosity of the order of 15 nm. Otherwise the filters would be rapidly clogged by the presence of such proteins of high molecular weight in the solution to be filtered. Finally, when viral inactivation by solvent-detergent treatment is carried out between chromatography on the anion exchange resin of step b) and chromatography on hydroxyapatite of step d), by purification on hydroxyapatite, it is possible to substantially remove the entirety of the solvent and of the detergent present in the protein concentrate loaded on the hydroxyapatite.

In a preferred embodiment, the method of the invention comprises at least one additional step for viral inactivation of the eluate resulting from step b) and/or of the eluate resulting from step d). Preferentially, the viral inactivation step carried out on the eluate resulting from step b) corresponds to a solvent-detergent treatment, preferably in the presence of a Tween (polysorbate 80)—TnBP mixture, preferably with a 1% (v/v) polysorbate 80—0.3% (v/v) TnBP mixture. In a particular embodiment, said at least one viral inactivation step is carried out in the form of a UV-C (Ultra Violet C) treatment, treatment with caprylate ions and/or by dry heating. Preferentially, the method of the invention may comprise a second step for virus removal carried out on the eluate resulting from step d) and corresponding to at least one nanofiltration, preferably at least on a filter with a porosity of 15 nm, preferably on a Planova 15N filter (Asahi). Enveloped viruses and those without envelope may thereby be removed.

The method of the invention may therefore comprise at least one viral inactivation step aiming at securing from a virological point of view the final product which is intended for therapeutic administration. A first viral inactivation step by treatment with a solvent-detergent mixture and allowing inactivation of enveloped viruses may be implemented at any stage of the method and preferably after purification on anion exchange resin. The solvent-detergent mixture used may correspond to any suitable mixture known to one skilled in the art and is preferably composed as indicated supra. The viral inactivation treatment by solvent-detergent is generally implemented for a period of a few hours (for example 7 hours), at substantially room temperature (for example 25+/−1° C.).

Moreover, the method of the invention may also comprise at least one viral removal step by nanofiltration on at least one filter with low porosity, for example on at least one filter with a porosity comprised between 15 nm and 100 nm. With such a nanofiltration step, it is more particularly possible to secure the final product with regard to non-enveloped viruses (viruses of the poliovirus or parvovirus type) and unconventional transmissible agents (of the prion type). Within the scope of the method of the invention, nanofiltration is carried out on at least one filter having a porosity of 15 nm, and preferably on at least one Planova 15N filter (Asahi). In a particular embodiment, nanofiltration is carried out on at least two filters having different porosity, preferably decreasing porosity. This nanofiltration is preferably carried out after chromatography on hydroxyapatite in so far that the presence of significant concentrations of proteins with high molecular weights (for example fibrinogen, fibronectin, or IgM) in the protein extract to be filtered generally leads to clogging of the filter, a fortiori when the method is implemented on an industrial scale. The PPSB concentrate resulting from the method comprising the two aforementioned viral inactivation steps proves to be compliant with international recommendations issued by the EMEA or the FDA as regards plasma and biotechnological products, insofar that it meets both the safety conditions required for non-enveloped viruses and for naked viruses.

In a preferred embodiment, the method of the invention comprises at least one additional diafiltration-ultrafiltration step after step b) and/or after step d). In a preferred embodiment, the method of the invention comprises two chromatography sub-steps on anion exchange resin. Then there is an additional step b2) consisting of applying the eluate of step b) on a second anion exchange resin and of eluting the concentrate of proteins dependent on vitamin K comprising proteins with high molecular weights. Preferentially, the second anion exchange resin is a resin of the DEAE-Sepharose type, and preferably DEAE-Sepharose FF (Amersham). A DEAE-Sepharose resin has the advantage of resisting to pressure as well as to the treatment with sodium hydroxide customarily used for sanitizing and regenerating the gel.

In a preferred embodiment, the method of the invention comprises a final additional formulation step, preferably by freeze-drying and/or adding pharmaceutically acceptable adjuvants or carriers. In an embodiment, the product obtained after formulation comprises 0.13 M NaCl, 2 g/L of arginine, 2 g/L of lysine, 3 g/L of sodium citrate, and has a pH from 6.9 to 7.1. In another embodiment, the product obtained after formulation comprises 10 g/L of arginine, 35 g/L of mannitol, and has a pH from 6.9 to 7.1. In another embodiment, the product obtained after formulation comprises 45 g/L of mannitol and has a pH from 6.9 to 7.1. In another embodiment, the product obtained after formulation comprises 1 g/L of sodium citrate, 35 g/L of mannitol, and has a pH from 6.9 to 7.1.

In a preferred embodiment, the method of the invention comprising a step for adding an inhibitor of thrombin, preferably anti-thrombin III or a mixture of anti-thrombin III and of heparin. The anti-thrombin may originate from human plasma or have a recombinant human origin, such as for example Atryn®, commercially distributed by GTC Biotherapeutics. This addition may be carried out after step b) and/or after step d). Preferably, the addition of the inhibitor of thrombin is carried out after the solvent-detergent treatment of the eluate resulting from step b), or before nanofiltration of the eluate resulting from step d). The cofactor II of heparin may also be used as an inhibitor of thrombin, in the same concentrations as those proposed for anti-thrombin.

By adding an inhibitor of thrombin, it is advantageously possible to prevent or limit the activation of prothrombin (FII) into thrombin during purification steps implemented during the method of the invention. The absence of thrombic activity in the concentrate of proteins dependent on vitamin K obtained by the method of the invention makes it compatible with use as a therapeutic or prophylactic drug in humans and allows satisfactory preservation of this concentrate.

Preferably, the anion exchange resin of step b) of the method of the invention has a positively charged group selected from diethylaminoethane (DEAE), polyethylene-imine (PEI) and quaternary aminoethane (QAE). This anion exchange resin is more preferably the DEAE-Sephadex A-500 distributed by GE Healthcare. With the chromatography of step b), it is possible to remove a portion (which may be significant) of the proteins constituted by albumin, immunoglobulins (except to, a certain extent, certain Ig(s) such as IgM), anti-thrombin III and alpha-antitrypsin. Recovery of the plasma proteins adsorbed on the anion exchange resin is performed by gradually increasing the ionic force. In a particular embodiment, the vitamin-K-dependent proteins obtained following step d) may then be purified independently with techniques well known to one skilled in the art, for example on an affinity gel.

The present invention also relates to the concentrate of prothrombic factor (proteins dependent on vitamin K obtainable by the method described above). This concentrate of proteins preferentially comprises the factors II, VII, IX and X, and has an IgM concentration of less than 0.1% (percentage based on the total protein level of the concentrate), a fibrinogen concentration of less than 0.1% (percentage based on the total protein level of the concentrate), a fibronectin concentration of less than 0.1% and a concentration of factors of the complement of less than 0.1%. Preferably, the concentrate of the invention also comprises the proteins C, S and Z, and has an average specific activity of FIX of at least 4 IU per mg of proteins.

The present invention finally relates to the use of the concentrate of prothrombic factor obtainable by the method of the invention, as a drug, and more particularly as a drug for treating and preventing hemorrhagic accidents related to the deficiency in factors dependent on vitamin K, such as a constitutional deficiency in factor II or in factor X, or to overdosage of anti-vitamin K. The method of the present invention is illustrated in a more detailed way by the examples which follow. These examples describe specific embodiments of the present invention and cannot be considered as restricting the scope of the latter.

### EXAMPLES

### Example 1

### Experimental Conditions Implemented for Purifying the Concentrate of Proteins Dependent on Vitamin K

A—Preparation of the Plasma Cryoprecipitate Supernatant.

As a starting material a plasma cryoprecipitate supernatant is used which is obtained by freezing-defreezing and centrifugation at 0-3° C. of frozen fresh plasma. Cryoprecipitation is achieved upstream from the plasma fractionation, at a temperature of less than 2° C., in order to separate the insoluble cryoprecipitate at a temperature of less than 4°, mainly composed of factor VIII, fibronectin and fibrinogen.

The cryoprecipitate is separated from the supernatant by continuous centrifugation at a temperature close to +4° C. The centrifugation supernatant is called a cryosupernatant. The cryosupernatant essentially contains albumin, immunoglobulins as well as the other coagulation factors including the vitamin-K-dependent factors, composed of Prothrombin (Factor II), Factor VII, Factor IX, Factor X, Protein C, Protein S and Protein Z.

B—Chromatography on an Anion Exchange Resin.

The following step consists of preparing a fraction enriched with vitamin-K-dependent factor after adsorption on a weak anion exchange gel, DEAE Sephadex A-50 (diethylamino-ethyl Sephadex). The cryosupernatant is heated up to a minimum temperature of +10° C. (optimally from +16 to 18° C.). This cryosupernatant may if necessary undergo clarifying filtrations on filters of 1 μm and then of 0.5 μm before its purification on DEAE-Sephadex gel. The volume of the purified cryosupernatant is conventionally from 2,000 to 3,000 liters. About 1.5 g of dry DEAE-Sephadex are used per liter of purified cryosupernatant.

Prior to purification, the DEAE-Sephadex powder is swollen (3 washes), with siftings of the gel on a stainless steel web after each wash. The preparation, the swelling and the equilibration of the DEAE-Sephadex are carried out in a 0.075 M sodium chloride solution in a container provided with a stirring blade and a tank bottom grid (sieve) which may let the liquid escape but retains the beads of DEAE-Sephadex. The swelling operation of DEAE-Sephadex is carried out at room temperature (15-25° C.).

The final equilibration of the gel is controlled by measuring the osmolarity of the effluent. The cryosupernatant at a preferential temperature of 17+/−1° C., is sent continuously onto the swollen and equilibrated DEAE-Sephadex at a flow rate of 400 kg per hour after equilibration of the supplied flow rates. The totality of the cryosupernatant is thus put into contact with the DEAE-Sephadex with continual stirring, allowing continuous binding of the factors dependent on vitamin K on the gel.

The gel is then washed three times with a buffer containing 0.2 M of NaCl, 10 mM of citric acid, at pH 7, in an amount of 140 l of buffer for 2,200 l of purified cryosupernatant. The elution of proteins dependent on vitamin K (and of proteins with high molecular weight which are co-purified with them) is achieved by means of a buffer of 2 M NaCl, 10 mM citric acid, at pH 7, in an amount of 75 l of buffer for 2,200 liters of purified cryosupernatant. The obtained protein fraction during the elution is then desalted by conventional means, i.e. by ultrafiltration with cassettes having a cut-off threshold of 10 kilodaltons and optionally 30 kilodaltons and dialysis against a 0.15 M NaCl, 10 mM citric acid buffer at pH 7.

The protein eluate resulting from the purification on DEAE-Sephadex will be called the “PPSB Intermediate Product 1” or “PPSB-PI-1” within the scope of the present application. At this stage of the purification method, it proves to be possible to freeze PPSB-PI-1 while waiting for the implementation of the other purification steps of the eluate resulting from DEAE-Sephadex at this stage.

C—Viral Inactivation by Solvent-Detergent Treatment.

The PPSB-PI-1 is then subject to viral inactivation by treatment with a solvent-detergent mixture, and more specifically by a treatment with (1% v/v) polysorbate 80—(0.3% v/v) tri n-butyl phosphate (TnBP). The viral inactivation treatment is carried out for a period of at least 6 hours at a temperature from 24 to 25° C.

Other detergents may be used as an alternative for polysorbate, such as cholate or octoxynol (Triton X100) in concentrations ranging from 0.5 to 2%, in the presence of TnBP, at a temperature from 15 to 30° C. but preferentially around 25° C. The minimum incubation time for viral inactivation is 4 hours but this incubation may be extended up to 12 hours. The generally applied pHs range from 6 to 8 and the total protein concentration from 10 to 40 g/L.

D—Chromatography on Hydroxyapatite HA.

D.1—Package of the Gel

The chromatography gel used is Macro prep ceramic hydroxyapatite (Biorad), having a particle size of 40 microns. The dry gel is suspended in a 0.4 M phosphate buffer at pH 6.8, and then transferred into a Pharmacia K50/30 column. The package is achieved at a flow rate of 100 cm/h. The amount used is 30 g of dry gel, which provides a column of 50 mL of packed gel. The column is rinsed with 5 column volumes of 2 M NaOH and stored in 2 M NaOH.

D.2—Preparation of PPSB-PI-1 to be Injected onto the Column.

The PPSB-PI-1 is, if necessary, de-frozen and virally inactivated for 3 hours in the presence of 1% polysorbate 80 and of 0.3% TnBP. The virally inactivated PPSB-PI-1 is then half diluted, optionally with a 20 mM benzamidine solution; and the pH of the solution is adjusted to 8 with 0.1 M NaOH.

D.3—Chromatography:

The column is connected to a Pharmacia UV detector provided with an industrial detection cell, and the optical density of the effluent is recorded at 280 nm. The gel stored in 2 M NaOH is washed with 5 volumes of pre-equilibration buffer (0.4 M potassium phosphate; pH 6.8). The column is then equilibrated with 15 volumes of equilibration buffer (0.01 M potassium phosphate; 0.075 M NaCl; 10 mM benzamidine (optional); pH 8). Next the PPSB solution is injected at a flow rate of 100 cm/h and the column is washed with the equilibration buffer until return to the baseline.

Pre-elution is carried out at the same flow rate with a pre-elution buffer (0.01 M potassium phosphate; 0.25 M NaCl; 10 mM benzamidine; pH 8 or 0.03 M potassium phosphate; 0.25 M NaCl; benzamidine 10 mM (optional); pH 8) and 5 column volumes of pre-eluate are collected. The gel is then washed with 15 volumes of the same buffer. The elution is carried out at the same flow rate with an elution buffer (0.5 M potassium phosphate; 0.075 M NaCl; 10 mM benzamidine (optional); pH 8) and 5 column volumes of eluate are collected. The gel is regenerated with 5 column volumes of 2 M NaOH and stored in 2 M NaOH.

E—Ultrafiltration and Dialysis

The eluate resulting from chromatography on hydroxyapatite is subject to ultrafiltration achieved on a 0.1 m² Sartorius ultrasart slice polysulfone cassette with a cut-off threshold of 10 kDa. The eluate is concentrated 3 times and dialyzed at constant volume against water purified for injection (pwi) until a resistivity of 70 ohms is obtained (the inlet pressure on the cassette is 0.5 bar and the ultrafiltration flow rate is 45 mL/min), and then dialyzed at constant volume against 5 volumes of dialysis buffer (3 g/L of trisodium citrate; 0.13 M of NaCl; 2 g/L of lysine; 2 g/L of arginine; pH 7). The product is then re-concentrated twice and the cassette is rinsed with the dialysis buffer so as to obtain a final volume equal to 80% of the initial volume. The product is finally frozen and stored at −40° C., and may, if need be, be subsequently filtered on a filter with a porosity of 15 nm.

F—Assay

The amount and/or concentrations of the coagulation Factor II (FII), Factor VII (FVII), Factor IX (FIX) and Factor X (FX) which make up the pro-thrombic complex (or PPSB) are assayed (by measuring the capability of inducing coagulation) and thrombic activity is measured. Also, the amount and/or the concentration of the C, S proteins is assayed by means of kits “Asserachrom® Total Protein S” and “Asserachrom® Protein C”, distributed by Stago.

The antigen assays of the factors VII, IX, X and of the protein Z are achieved by ELISA by means of the kits. “Asserachrom® VII:Ag”, “Asserachrom® IX:Ag”, “Asserachrom® X:Ag”, “Asserachrom® Protein Z” distributed by Stago. The amounts and/or the concentrations of polysorbate 80 and of TnBP are measured.

### Example 2

### Experimental Results

A: Study of the Capacity of the Column.

The capacity of the hydroxyapatite column was tested with doses of 3, 5, 7 and 9 mL of virally inactivated PPSB-PI-1 per mL of gel, under the same experimental conditions as those described above. No pre-elution was carried out. The yield calculated for each factor corresponds to the ratio of the total amount of coagulating units in the eluate resulting from hydroxyapatite over the total amount of coagulating units in PPSB-PI-1. The yields obtained according to the load are detailed in the following Table I:

TABLE 1
Elution yields in factors II, IX, VII and X depending on
the load on the gel.

| Load of the column | | | | |
|---|---|---|---|---|
| mL of PPSB-PI-1/mL | FII yields | FVII yields | FIX yields | FX yields |
| of gel | (%) | (%) | (%) | (%) |
| 3 | 88 | 103 | 112 | 92 |
| 5 | 91 | 111 | 111 | 79 |
| 7 | 108 | 88 | 106 | 69 |
| 9 | 93 | 85 | 96 | 57 |

These data are graphically illustrated in FIG. 1. A clear decrease in the binding of FX versus the load is observed.

The percentage of non-bound factor calculated for each factor corresponds to the ratio of the total amount of coagulating units in the non-bound fraction to the total amount of coagulating units in the initial PPSB-PI-1. The percentages of non-bound factor are summarized in Table II.

TABLE II
Amount of factors II, IX, VII and X not retained on the
gel versus the load.

| Load of the | | | | |
|---|---|---|---|---|
| column | | | | |
| mL of | Non-bound | Non-bound | Non-bound | Non-bound |
| PPSB-PI-1/mL | FII | FVII | FIX | FX |
| of gel | (%) | (%) | (%) | (%) |
| 3 | <1 | <1 | <1 | 5 |
| 5 | 3 | <1 | <1 | 14 |
| 7 | 11 | 5 | <1 | 26 |
| 9 | 18 | 8 | <1 | 35 |

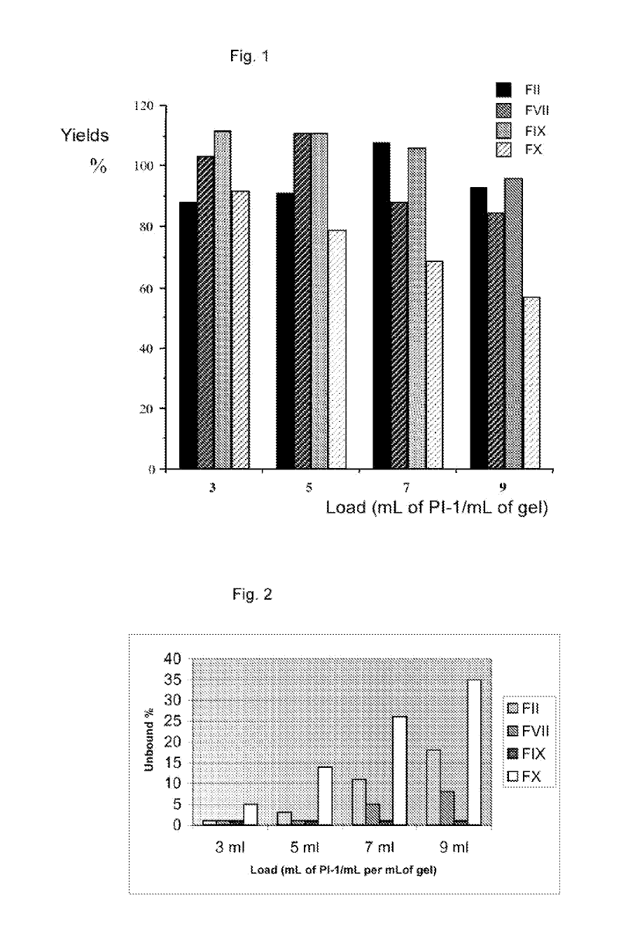

These data are graphically illustrated in FIG. 2. In this last figure, it appears much clearer that the FII and the FX are the factors which bind the less on the hydroxyapatite column.

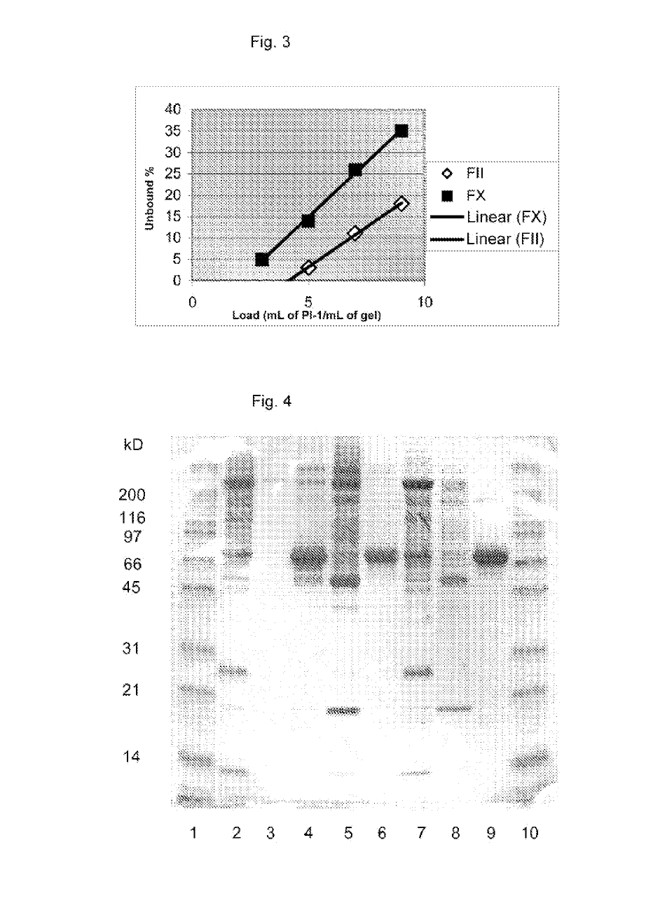

FIG. 3 moreover shows that for both of these factors (FII and FX), the percentage of non-bound factor varies linearly with the load. The 5 mL load of PPSB-PI-1 per mL of gel for which the binding of FII and FX is still acceptable, was retained.

B—Influence of Pre-Elution

With the purpose of removing as many contaminating proteins as possible, pre-elution with a 30 mM phosphate buffer was tested. Such a pre-elution is preferably carried out with a 20 to 40 mM phosphate buffer, and more preferentially of 30 mM, in so far that elution of the factors II and VII has been observed from a phosphate concentration of 50 mM. The operating conditions are identical with those described above and the protein load used is 5 mL of PPSB-PI-1 per mL of gel.

No coagulation factor was able to be detected in the pre-eluates. The yields are therefore not affected by this pre-elution, as shown by the following results:

TABLE III
Amount of factors II, XII, VII and X in the eluates
versus the type of achieved pre-elution type.

| Test | | FII:C | FVII:C | FIX:C | FX:C |
|---|---|---|---|---|---|
| number | Pre-elution | (%) | (%) | (%) | (%) |
| 3 | 0.25M NaCl | 83 | 86 | 86 | 68 |
| 4 | 0.25M NaCl | 88 | 103 | 112 | 92 |
| 5 | 0.25M NaCl, 30 mM | 95 | 80 | 93 | 69 |
| | phosphate | | | | |
| 6 | 0.25M NaCl, 30 mM | 80 | 81 | 79 | 72 |
| | phosphate | | | | |

TABLE IV
Amount of co-purified proteins removed during the
phosphate pre-elution.

| | Total proteins in | Total proteins |
|---|---|---|
| | 30 mM phosphate | in eluate |
| Test number | pre-eluate (mg) | (mg) |
| 3 (no phosphate | 5 | 1218 |
| pre-elution) | | |
| 4 (no phosphate | 0 | 916 |
| pre-elution) | | |
| 5 | 44 | 964 |
| 6 | 68 | 897 |
| 7 | 52 | 1078 |
| 8 | 58 | 1013 |
| 9 | 57 | 1061 |
| 10 | 62 | 1061 |
| 11 | 62 | 1125 |
| 12 | 68 | 1166 |

The pre-elution carried out with a 30 mM phosphate buffer further allows removal of a large number of accompanying proteins and notably proteins of high molecular weight (100 to 200 kD) as well as shown by the SDS PAGE gel (see FIG. 4)

E—Influence of the Addition of Antithrombin and Heparin

In order to maintain low thrombic activity in the protein extract during purification, purified antithrombin at a concentration of 0.5 U/mL (preferably at a concentration from 0.1 to 0.04 unit of antithrombin per unit of FIX) and heparin at 2 U/mL are added into the hydroxyapatite chromatography eluate before ultrafiltration. The experimental conditions are the same as the ones described above, with pre-elution in a 30 mM phosphate buffer. The load of the column was 5 mL of PPSB per mL of gel.

E1—FII, FX, FVII and X Activities in Dialyzed Eluates after Purification on Hydroxyapatite.

TABLE V
Activities of factors II, IX, VII and X and thrombic activity in
the eluate after purification on hydroxyapatite.

| | | | | | Thrombic | Thrombic |
|---|---|---|---|---|---|---|
| | FII: C | FVII: C | FIX: C | FX: C | activity | activity |
| | U/mL | U/mL | U/mL | U/mL | 6 h 37° C. | 25 h 20° C. |
| Test | | | | | | |
| number | | | | | | |
| 25 | 19 | 8.4 | 23 | 19 | >6 h | >24 h |
| 26 | 19 | 8.5 | 22 | 19 | >6 h | >24 h |
| 27 | 21 | 8.5 | 17 | 21 | >6 h | >24 h |
| 28 | 20 | 9.5 | 17 | 22 | >6 h | >24 h |
| Average | 20 | 9 | 20 | 20 | | |
| standard | | | | | | |
| deviation | 1.0 | 0.5 | 3.2 | 1.5 | | |
| C.V. | 4.8 | 5.9 | 16.2 | 7.4 | | |
| PI-1 | 35 | 16 | 27 | 48 | >6 h | >24 h |

The conducted tests are reproducible between them and the thrombic activities measured at 6 h and at 24 h are quasi zero, and are consequently compliant for subsequent therapeutic use of the concentrate of proteins depending on vitamin K (a thrombic activity of 6 hours and more corresponds to very small amounts of thrombin, much less than 0.001 NIH units).

E2—FII, FX, FVII and X Yields in the Dialyzed Eluate after Purification on Hydroxyapatite.

TABLE VI
Yields in factors II, IX, VII and X and thrombic activity
in the dialyzed or ultrafiltered eluate after purification on hydroxyapatite.

| Test number | FII:C % | FVII:C % | FIX:C % | FX:C % |
|---|---|---|---|---|
| 25 | 57 | 59 | 72 | 59 |
| 26 | 64 | 63 | 63 | 60 |
| 27 | 76 | 57 | 77 | 65 |
| 28 | 85 | 60 | 70 | 71 |
| Average | 71 | 60 | 71 | 64 |
| standard | 12.4 | 2.5 | 5.8 | 5.5 |
| deviation | | | | |
| C.V. | 17.7 | 4.2 | 8.2 | 8.6 |

The yields are of the order of 70% for the factors II and IX and of the order of 60 and 64% on average for the factors VII and X.

TABLE VII
Yields in factors II, IX, VII and X after ultrafiltration of
the eluate resulting from the hydroxyapatite.

| Test number | FII:C % | FVII:C % | FIX:C % | FX:C % |
|---|---|---|---|---|
| 25 | 66 | 74 | 77 | 77 |
| 26 | — | — | — | — |
| 27 | 97 | 79 | 72 | 89 |
| 28 | 85 | 60 | 70 | 71 |
| Average | 83 | 71 | 73 | 79 |
| standard | 15.6 | 9.8 | 3.6 | 9.2 |
| deviation | | | | |
| C.V, | 18.9 | 13.9 | 4.9 | 11.6 |

The yields for the whole of the factors after ultrafiltration are of the order of 70 to 80%.

TABLE VIII
Specific factor II, X, VII and IX activities in the
dialyzed eluate after hydroxyapatite.

| | | | | | Total |
|---|---|---|---|---|---|
| Test | FII:C | FVII:C | FIX:C | FX:C | proteins |
| number | U/mg | U/mg | U/mg | U/mg | mg/mL |
| 25 | 6.3 | 2.8 | 7.7 | 6.8 | 3 |
| 26 | 5.7 | 2.7 | 4.9 | 6.3 | 2.9 |
| 27 | 6.8 | 2.7 | 5.5 | 6.8 | 3.1 |
| 28 | 5.7 | 2.7 | 4.9 | 6.3 | 3.5 |
| Average | 6.1 | 2.7 | 5.8 | 6.6 | 3.1 |
| standard | 0.5 | 0.1 | 1.3 | 0.3 | 0.3 |
| deviation | | | | | |
| C.V. | 8.7 | 1.8 | 23.1 | 4.4 | 8.4 |
| PPSB-PI-1 | 1.2 | 0.6 | 1 | 1.2 | 35 |

The specific activities calculated for each factor are increased by about five times relatively to a concentrate of FII, FVII, FIX and FX obtained by applying a chromatography on anion exchange resin in the place of the hydroxyapatite of the invention.

F—Conclusion on the Purification Step on Hydroxyapatite

The binding of the coagulation factors depending on vitamin K appears to be more specific on surfaces based on calcium phosphate (of the hydroxyapatite type) as compared with the ion exchange gel, which explains the purity of the obtained product. The analysis of the recovered proteins after elution on hydroxyapatite gel shows that the proteins comprised in the protein concentrate of interest have a molecular weight comprised between 75 and 50 kDa, which corresponds to the molecular weight of the proteins dependent on vitamin K, and in particular to the molecular weight of the different coagulation factors composing the prothrombic complex.

Table IX below establishes the list of proteins present in a protein concentrate obtained by purification on an anion exchange resin after viral inactivation (and as a replacement for purification on hydroxyapatite of the invention). It should be noted that in such a concentrate, the sum of the proteins depending on vitamin K (FII, FVII, FIX, FX, protein C, protein S, protein Z) represents 17% of the total proteins.

TABLE IX
List and relative proportions of the proteins present in
a concentrate resulting from single purification on an anion
exchange resin or from purification comprising a first anion
exchange resin and a second anion exchange resin as a
replacement for chromatography on hydroxyapatite of the invention.

| Proteins | % | MW |
|---|---|---|
| IgM | 0.30 to 0.40 | 900 |
| C 4 bp | 1.60 to 2 | 590 |
| Fibronectin | 0.45 to 0.65 | 440 |
| Fibrinogen | 0.65 to 1 | 330 |
| C 4 | 9 to 13 | 206 |
| C 3 | 0.55 to 0.75 | 180 |
| C 3 c | 0.45 to 0.65 | 180 |
| C 5 | 0.05 to 0.1 | 180 |
| Protein S | 0.90 to 1.15 | 75 |
| Prothrombin (FII) | 12 to 15 | 68.7 |
| Albumin | 2.10 to 2.5 | 68 |
| Antithrombin III | 0.05 to 1.5 | 65 |
| Factor X | 1.25 to 1.75 | 59 |
| Protein C | 0.20 to 0.50 | 57 |
| Factor IX | 0.15 to 0.45 | 55.4 |
| Protein Z | 0.05 to 0.20 | 55 |
| Factor VII | 0.01 to 0.05 | 50 |

On the contrary, the protein concentrate resulting from the method of the invention and notably obtained after the chromatography step on hydroxyapatite contains a proportion of proteins dependent on vitamin K (FII, FVII, FIX, FX, protein C, protein S, protein Z) of the order of 90 to 95% of the total proteins. The concentrate which results from this chromatography on hydroxyapatite has a specific activity increased by 5 to 7 times relatively to the intermediate product 1 resulting from the chromatography on anion exchange resin. Additional measurements moreover show that chromatography on hydroxyapatite further allows efficient removal of the polysorbate 80 and of TnBP used during the viral inactivation step.

Finally, the addition of antithrombin III and of heparin in the hydroxyapatite chromatography eluate gives the possibility of systematically obtaining a product without any thrombic activity at 24 h. Finally, the removal of most of the contaminating proteins and notably of the contaminating proteins of high molecular weights allows filtration of the product on membranes with a porosity of 15 nm by using acceptable filtration surfaces (about 10 liters of product per square meter of membrane).

G—Nanofiltration of the Concentrate Obtained after Chromatography on Hydroxyapatite

G1—Experimental Implementation of Nanofiltration

The filters used are filters with reference Planova 15 N (Asahi). The filters used consist of hollow fibers in hydrophilic copper-ammonium cellulose, the pore rated size of which is 15+/−2 nm. The equilibration buffer of the filters consists of 0.13 M sodium chloride, 3 g/L of tri-sodium citrate 2H₂O, 2 g/L of lysine HCl, 2 g/L of arginine HCl and purified water for injections (pwi). The buffer is adjusted to a pH of 7.0+/−0.05, to a resistivity of 75 Ω·cm, and to osmolality of 314 mosmol/kg of H₂O, at a temperature of 20 to 25° C.

The filters are prepared individually, rinsed with purified water for injections (pwi) under a pressure of the order of 500 mbars. The integrity of the filter is controlled before use after rinsing with purified (pwi) for injections. By conducting the air leak test or “leakage test”, the absence of an air passage through the fibers is checked in the external jacket under an air pressure of 1,000+/−50 mbars. (SOP of an integrity test for Asahi Planova Filters). Before filtering the solution, the filter is equilibrated by means of the formulation buffer. In a particular embodiment, the formulation buffer consists of 0.13 M NaCl, 2 g/L of arginine, 2 g/L of lysine, 3 g/L of sodium citrate, and has a pH from 6.9 to 7.1. In another embodiment, the formulation buffer consists of 10 g/L of arginine, 35 g/L of mannitol, and has a pH of 6.9 to 7.1. In another embodiment, the formulation buffer is composed of 45 g/L of mannitol and has a pH of 6.9 to 7.1. In another embodiment, the formulation buffer is composed of 1 g/L of sodium citrate, 35 g/L of mannitol, and has a pH from 6.9 to 7.1.

The pH and the resistivity of the 15 nm filtrate are checked (pH 7.0+/−0.1—Osmolality 314+/−10 mosmol/kg H₂O). A flask of eluate resulting from the purification on ceramic hydroxyapatite described above and optionally dialyzed is de-frozen in a water bath at 37° C.+/−2° C. Pre-filtration is optionally achieved on a 0.2 μm cellulose tri-acetate filter (Sartolab P—Sartorius) before filtering on the Planova 15N 15 nm filter.

The filtration of the eluate is carried out under a constant compressed air pressure of 500+/−50 mbars. The pressure measurement is carried out at the inlet of the 15 nm filter by means of a digital pressure gauge. The eluate resulting from the filtration on a 15 nm filter is collected at the low outlet of the filter in a flask placed on scales. Readouts of the filtered weight are carried out at regular time intervals in order to determine the filtration flow rate. The achieved filtration is frontal without any re-circulation.

At the end of the filtration, an air leakage test (“leakage test”) is conducted after filling the external jacket of the filter in order to check the integrity of the membrane and to validate the filtration step. In order to test the reproducibility of the nanofiltration step, the tests are conducted by applying standardized operating conditions detailed in Table X.

TABLE X
Operating conditions and parameters applied during
15 nm nanofiltration.

| Operating parameters | Values |
|---|---|
| Temperature ° C. | 20 +/− 2 |
| Applied pressure mbars | 500 +/− 50 |
| Total proteins g/L | 5.0 +/− 1.0 |
| Factor IX:C Total IU | About 3,200 |
| Average protein load g/m² | 50 +/− 10 |
| Volumetric load l/m² | 10.0 +/− 1 |

After nanofiltration, the amount and/or the concentration of the coagulation factors FII, FVII, FIX and FX is measured by conventional techniques known to one skilled in the art. The same applies for the determination of the total protein levels.

G.2—Results Obtained after Nanofiltration of the Eluate Resulting from Hydroxyapatite.

G.2.1—Tracking the Nanofiltration Parameters

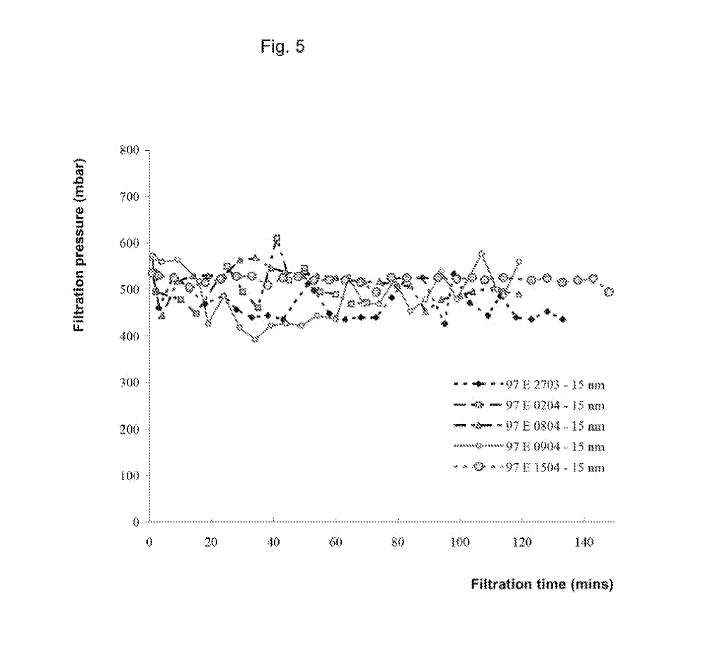

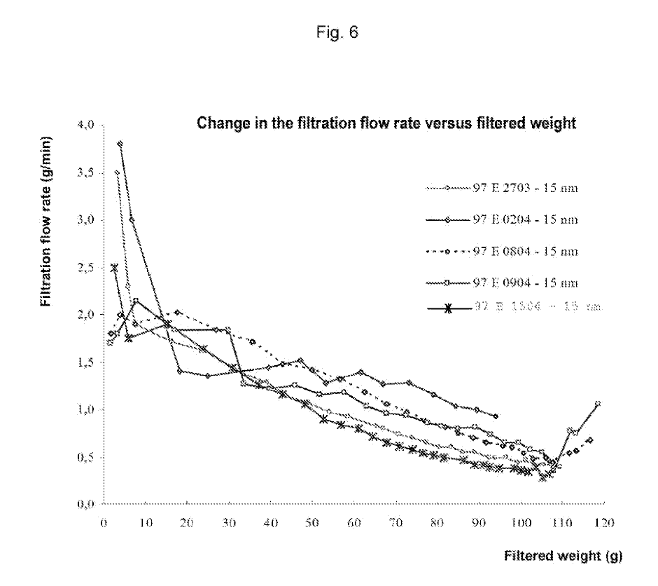

The filtration pressure was maintained within an average of 500+/−100 mbars during filtration tests (see FIG. 5). The change in the filtration flow rate is measured during nanofiltration (see FIG. 6). A regular reduction in the filtration flow rate is observed depending on the filtered weight. A similar profile is obtained for all the conducted tests.

TABLE XI
Tracking the nanofiltration flow rates.

| | Initial flow | | |
|---|---|---|---|
| Nanofiltration test | rate | Final flow rate | |
| number | g/min | g/min | Ratio % |
| 97 E 2703 | 2.3 | 0.4 | 17.4 |
| 97 E 0204 | 3.0 | 0.9 | 30.0 |
| 97 E 0804 | 2.0 | 0.6 | 30.0 |
| 97 E 0904 | 1.8 | 0.6 | 33.3 |
| 97 E 1504 | 1.8 | 0.3 | 16.7 |

According to the recommendations of the supplier of the Asahi filter, the final filtration flow rate should be greater than 10% of the initial flow rate. For a flow rate ratio of less than 10% the filter is considered as being clogged, in particular for pores of smaller diameter. Continuation of the filtration may actually promote the passage of potential viruses through the wider pores of the membrane. The ratio of the final/initial nanofiltration flow rates is compliant for all the conducted tests.

TABLE XII
Tracking the filtration parameters.

| | | | Average flow | |
|---|---|---|---|---|
| Nanofiltration | Filtration time | Capacity | rate | Capacity |
| test number | Mins | L/m² | L/hour/m² | g/m² |
| 97 E 2703 | 138 | 11.4 | 4.9 | 50.2 |
| 97 E 0204 | 70 | 9.7 | 8.0 | 42.7 |
| 97 E 0804 | 89 | 10.0 | 6.8 | 49.0 |
| 97 E 0904 | 99 | 10.5 | 6.4 | 52.5 |
| 97 E 1504 | 148 | 10.7 | 4.3 | 51.4 |

The average filtration time is of the order of 2 hours. The average volumetric capacity is of the order of 10 L/m² of membrane, which corresponds to a capacity of about 47.9+/−4.7 g of proteins per m².

G.2.2—Balance of Total Proteins and of Coagulation Factors after Nanofiltration

TABLE XIII
Total protein balance.

| Nanofiltration | Volume V₀ | Total proteins | | 15N permeate | Total proteins | | Yield |
|---|---|---|---|---|---|---|---|
| test number | mL | g/L | mg | mL | g/L | mg | % |
| 97 E 2703 | 120.0 | 4.6 | 552 | 114.6 | 4.4 | 504 | 95.7 |
| 97 E 0204 | 102.0 | 4.8 | 490 | 108.7 | 4.4 | 478 | 97.7 |
| 97 E 0804 | 112.0 | 6.4 | 717 | 116.7 | 5.9 | 689 | 96.0 |
| 97 E 0904 | 110.0 | 5.5 | 605 | 121.4 | 5.0 | 607 | 100.3 |
| 97 E 1504 | 112.0 | 4.8 | 538 | 112.0 | 4.8 | 538 | 94.0 |
| Average/standard | 111.2 +/− 6.4 | 5.2 +/− 0.7 | 580 +/− 86 | 114.7 +/− 4.8 | 4.9 +/− 0.6 | 563 +/− 85 | 96.7 +/− 2.4 |
| deviation | | | | | | | |

Good total protein nanofiltration yields are noted with values of more than 90%. The tests are conducted from raw materials from different batches and prove to perfectly reproducible.

TABLE XIV
Coagulation factors balance during the 15 nm nanofiltration step.

| | FII: C | | FVII: C | | FIX: C | | FX: C | |
|---|---|---|---|---|---|---|---|---|
| | IU/mL | | IU/mL | | IU/mL | | IU/mL | |
| Nanofiltration | Before | After | Before | After | Before | Filtrate | Before | Filtrate |
| test number | 15N | 15N | 15N | 15N | 15N | 15N | 15N | 15N |
| 97 E 2703 | 30 | 36 | 16.0 | 15.0 | 25 | 23 | 24 | 22 |
| 97 E 0204 | 32 | 28 | 19.0 | 18.0 | 27 | 25 | 30 | 27 |
| 97 E 0804 | 39 | 39 | 30.0 | 26.0 | 32 | 35 | 39 | 36 |
| 97 E 0904 | 34 | 39 | 20.0 | 17.0 | 31 | 26 | 32 | 29 |
| 97 E 1504 | 30 | 30 | 19.0 | 19.0 | 29 | 29 | 29 | 29 |
| Average/ | 33 +/− 3.7 | 34.4 +/− 5.1 | 20.8 +/− 5.4 | 19.0 +/− 4.2 | 28.8 +/− 2.9 | 27.6 +/− 4.7 | 30.8 +/− 5.4 | 28.6 +/− 5.0 |
| standard | | | | | | | | |
| deviation | | | | | | | | |

Good reproducibility of the different tests is noted for all the coagulation factors.

The chromatography step on ceramic hydroxyapatite allows good recovery for the whole of the factors dependent on vitamin K. It is noted that the concentrations before and after 15 nm nanofiltration are very close, thereby demonstrating a high filtration yield for the four coagulation factors.

TABLE XV
Time-dependent change in the specific activity during 15 nm nanofiltration.

| | S.A. FII | | S.A. FVII | | S.A. FIX | | S.A. FX | |
|---|---|---|---|---|---|---|---|---|
| | IU/mg | | IU/mg | | IU/mg | | IU/mg | |
| Nanofiltration | Before | After | Before | After | Before | After | Before | After |
| test volume | 15N | 15N | 15N | 15N | 15N | 15N | 15N | 15N |
| 97 E 2703 | 6.5 | 8.2 | 3.5 | 3.4 | 5.4 | 5.2 | 5.2 | 5.0 |
| 97 E 0204 | 6.7 | 6.4 | 4.0 | 4.1 | 5.6 | 5.7 | 6.3 | 6.1 |
| 97 E 0804 | 6.1 | 6.6 | 4.7 | 4.4 | 5.0 | 5.9 | 6.1 | 6.1 |
| 97 E 0904 | 6.2 | 7.8 | 3.6 | 3.4 | 5.6 | 5.2 | 5.8 | 5.8 |
| 97 E 1504 | 6.3 | 6.3 | 4.0 | 4.0 | 6.0 | 6.0 | 6.0 | 6.0 |
| Average/ | 6.4 +/− 0.24 | 7.1 +/− 0.88 | 4.0 +/− 0.47 | 3.9 +/− 0.44 | 5.5 +/− 0.36 | 5.6 +/− 0.38 | 5.9 +/− 0.42 | 5.8 +/− 0.46 |
| standard | | | | | | | | |
| deviation | | | | | | | | |

The determined specific activities for all the coagulation factors are of the same order for all the conducted tests before and after nanofiltration. The determined specific activity relatively to the factor IX is greater than 5 for all the conducted tests.

TABLE XVI
Yield in coagulation factors after 15 nm nanofiltration.

| Nanofiltration | | | | |
|---|---|---|---|---|
| test | | | | |
| number | FII:C % | FVII:C % | FIX:C % | FX:C % |
| 97 E 2703 | 120.0 | 100.0 | 104.5 | 95.7 |
| 97 E 0204 | 93.2 | 101.0 | 98.7 | 95.9 |
| 97 E 0804 | 104.2 | 90.3 | 114.0 | 96.2 |
| 97 E 0904 | 126.6 | 93.8 | 92.6 | 100.0 |
| 97 E 1504 | 84.8 | 107.5 | 98.4 | 95.2 |
| Average/ | 105.8 +/− | 98.5 +/− 6.7 | 101.6 +/− 8.1 | 96.6 +/− 1.9 |
| standard | 17.6 | | | |
| deviation | | | | |

For each conducted test a yield of the same order (and greater than 90%) is observed for all the coagulation factors.

G.2.3—Determination of Thrombic Activity

The determination of the thrombic activity is achieved on an automatic apparatus detecting the occurrence of a clot by opacification of the sample. The conducted analysis corresponds to a coagulation test, the sensitivity of which allows detection of a small amount of residual thrombin in a sample. The result is compliant if absence of coagulation is obtained after 6 hours at 37° C. and after 24 hours at 24° C.

No thrombin generation is observed during nanofiltration of the eluate resulting from the hydroxyapatite. The determination of thrombic activity indicates an absence of coagulation after 6 hours at 37° C. However, the conducted tests show that an onset of formation of a clot may be observed at time 24 hours, this result being confirmed by a test traditionally conducted in a water bath.

Experiments were carried out in order to determine whether the addition of an inhibitor of proteases, such as for example antithrombin III, causes the disappearance of the residual thrombic activity observed at 24 hours. The determination of the protease activities was achieved by spectrophotometry, by using specific chromogenic substrates. The hydrolysis of a specific chromogenic substrate by a protease is actually accompanied by the release of a molecule of yellow color detected at 405 nm, the occurrence rate of which is proportional to the concentration of the enzyme of the tested solution.

TABLE XVII
Determination of thrombic activity with the
chromogenic substrate S 2238

| | | S 2238 + i | | S 2238 + |
|---|---|---|---|---|
| | S 2238 | 2581 | Activity IIa | AT-III + |
| Steps | U OD | U OD | Δ U OD | Heparin |
| PPSB-PI-1 97 E 1106 | 0.0172 | 0.0142 | 0.0030 | 0.0149 |
| Dialyzed HA eluate | 0.0314 | 0.0200 | 0.0114 | 0.0339 |
| 97 E 1106 | | | | |
| 15 nm filtrate | | | | |
| 97 E 2506 | 0.0065 | 0.0020 | 0.0045 | 0.0014 |
| 97 E 1007 | 0.0075 | 0.0015 | 0.0060 | 0.0021 |
| 97 E 1607 | 0.0090 | 0.0021 | 0.0069 | 0.0017 |
| 97 E 1707 | 0.0103 | 0.0018 | 0.0085 | 0.0018 |

The substrate S2238 is a specific substrate of thrombin. It is noted that in the absence of an inhibitor, residual activity of the thrombin type is observed in all the tested samples. This activity is substantially inhibited by addition of the inhibitor of thrombin i 2581 and equivalently in the presence of a mixture of antithrombin III (AT-III) and heparin. The thrombin may therefore be efficiently neutralized by its physiological inhibitor AT-III in the presence of heparin.

The residual thrombic activity measured by the test of the thrombic activity with the chromogenic substrate corresponds to an activity of less than 0.1 IU/mL in 15 nm filtrates. Aprotinin also exhibits a good efficiency for inhibiting residual proteases. However, the bovine origin of this inhibitor does not allow it to be used within the scope of purification of products intended for therapeutic use in humans.

G.2.4—Characterization of the Protein Concentrate by SDS Page Electrophoresis.

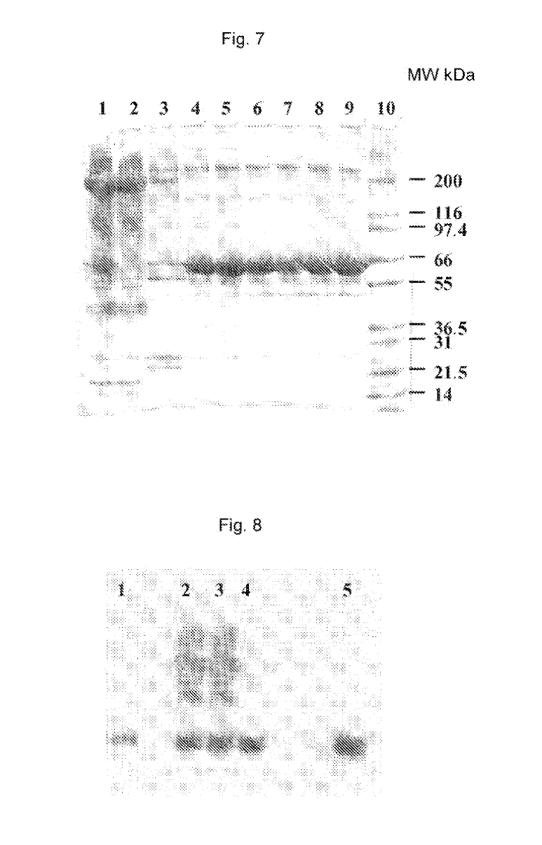

As this may be seen in FIG. 7, the majority of the proteins of high molecular weight have been removed in the non-adsorbed fraction from chromatography on HA ceramic hydroxyapatite. No notable composition difference is noted in the 15 nm nanofiltration step, the 15 nm filtrate proving to be similar to the protein concentrate before nanofiltration in every point. The major 66 kDa band essentially corresponds to prothrombin which represents by itself about 60% of the total proteins of the protein concentrate.

G.2.5—Characterization of the Factor IX by Immunoblot

An immunoblot is produced after electrophoresis on homogeneous 10% SDS Page gel without any reducing agent. After transfer on nitrocellulose and saturation with albumin, a contact with an anti-factor IX monoclonal primary antibody (Sigma Ref. F1020) is achieved. The marking by an anti-mouse secondary antibody marked with peroxidase (BioRad) is carried out before development by an ECL technique on autoradiography film (Pierce). The results are shown in FIG. 8. The presence of non-specific bands is observed in the wells corresponding to PI-1.

On the contrary, the dialyzed eluate from the ceramic hydroxyapatite column only exhibits a single homogeneous band. Moreover no visible difference is noted after 15 nm nanofiltration, nor with the factor IX HP used as a control.

G.2.6—Conclusion

The nanofiltration on Planova Asahi 15 nm filter of the dialyzed eluate resulting from chromatography on ceramic hydroxyapatite was carried out in a reproducible way by observing standardized operating conditions. A decrease in the flow rate proportional to the filtered weight is noted in a reproducible way. The average volumetric capacity of the filter is of the order of 10 L/m² of membrane, which corresponds to an average capacity of 47.9+/−4.7 g of proteins for the conducted tests.

The total protein balance gives an average yield of 91.1+/−14.0%, comparable with the yield in coagulation factors of 99.4+/−22.2 for FII:C, 91.5+/−18.2 for the factor VII:C, 95.8+/−16.1 for the factor IX:C and 90.5+/−15.1 for the factor X:C. The specific activity for the various factors is of the same order before and after nanofiltration. No notable difference is noted before and after 15 nm filtration by characterization with SDS Page electrophoresis.

H—Tests for Optimizing the Stability of the Solution During the Manufacturing

Complementary tests were conducted by varying different parameters with the purpose of obtaining an eluate resulting from the hydroxyapatite which may be filtered on 15 nm and have a very low or inexistent thrombic activity content.

TABLE XVIII
List of the tests conducted for stabilising the
ceramic hydroxyapatite eluate.

| | Formu- | | Benzam- | | | |
|---|---|---|---|---|---|---|
| Test | lation | Raw | idine | AT III | Heparin | Thrombic |
| number | number | material | mmol/L | U/mL | IU/mL | activity |
| 97 E 1806 | a | Frozen | 50 | — | 2.0 | Compliant |
| 97 E 1007 | a | Fresh | — | 2.0 * | 5.0 | Compliant |
| 97 E 2312 | b | Fresh | — | 0.5 ** | 2.0 | Compliant |
| 97 E 3012 | | | | | | |
| 98 E 0801 | b | Fresh | — | 0.5 ** | — | Compliant |
| 98 E 1301 | | | | | | |

* AT-III added at the moment of the solvent-detergent treatment before chromatography.
** AT-III added before ultrafiltration.

Formulation a: 0.13 M NaCl—0.010 M sodium citrate, 2.0 g/L of Lysine HCl, 2.0 g/L of Arginine HCl, pH 7.0+/−0.1. Formulation b: 0.13 M NaCl, 0.010 M sodium citrate, pH 7.0+/−0.1. In order to be able to compare the results of the tests with each other, the parameters for conducting the nanofiltration step were not modified.

In the 97 E 1806 test, 50 mM benzamidine was added in the buffers and 2 IU/mL heparin was added before the 15 nm nanofiltration step. The protein concentrate resulting from nanofiltration on a filter with a porosity of 15 nm, does not have any thrombic activity. During the test 97 E 1007, 5 IU/mL heparin and 2 U/mL AT-III were added at the moment of the solvent-detergent treatment and before the chromatography step on hydroxyapatite, good filterability of 12.5 L/m² and absence of coagulation (and therefore of thrombic activity) are observed. In the 97 E 2312 and 97 E 3012 tests, 2 IU/mL heparin and 0.5 IU/mL AT-III are directly added into the eluate resulting from chromatography on hydroxyapatite before the ultrafiltration step. The protein concentrate resulting from nanofiltration on a filter with a porosity of 15 nm, does not have any thrombic activity. In the 98 E 0801 and 98 E 1301 tests, 0.5 IU/mL AT-III is added in the absence of heparin into the eluate resulting from the chromatography on hydroxyapatite before ultrafiltration. In both of these cases, the protein concentrate resulting from nanofiltration on a filter with a porosity of 15 nm does not exhibit any thrombic activity.

Antithrombin III therefore forms a good inhibitor of thrombic activity of the protein concentrate containing the prothrombic complex of the invention. Heparin further seems to act as a cofactor of AT-III and to potentialize the inhibitory activity of the latter. The best efficiency of antithrombin is thus obtained when the latter is added into the hydroxyapatite chromatography eluate. Indeed, it appears that antithrombin only binds very little onto hydroxyapatite.

I—Comparison between the protein concentrate obtained by the method of the invention and a concentrate obtained by a method comprising chromatography on an ion exchange resin as a replacement for chromatography on hydroxyapatite.

TABLE XIX
Comparison of the composition of a purified PPSB
with ion exchange chromatography as a replacement for
chromatography on hydroxyapatite and of the 15N
nanofiltered concentrate according to the invention.

| | Concentrate obtained by | |
|---|---|---|
| | a method comprising | |
| | anion exchange | Nanofiltered |
| | chromatography as a | concentrate obtained |
| | replacement for | by the method of the |
| Characteristics | hydroxyapatite | invention |
| Total proteins | 32.0 +/− 7.5 | 5.0 +/− 0.6 |
| mg/mL | | |
| SA IU FIX/mg | 0.8 +/− 0.2 | 5.9 +/− 0.4 |
| FII IU/mL | 40.0 +/− 8 | 35.0 +/− 4.8 |
| FVII IU/mL | 26.5 +/− 8.5 | 19.0 +/− 3.8 |
| FIX IU/mL | 25.5 +/− 5.5 | 27.8 +/− 4.2 |
| FX IU/mL | 40 +/− 8 | 29.2 +/− 4.7 |
| Thrombic activity at | Absent | Absent |
| 37° C. at 6 hours | | |
| Thrombic activity at | Absent | Absent |
| 25° C. at 24 hours | | |
| Heparin IU/IU FIX | 0.1-0.25 | 0.1-0.25 |
| Antithrombin IU/mL | Absent | 0.5-1 |

The results of Table XIX show that chromatography on ceramic hydroxyapatite carried out within the scope of the present invention gives the possibility of obtaining a considerably larger purification level of the proteins dependent on vitamin K than that which would be obtained with the use of a second anion exchange resin as a replacement for the hydroxyapatite of the invention. Moreover, the whole of the factors depending on vitamin K represent 70 to 80% of the total proteins for the nanofiltered concentrate obtained by the method of the present invention, versus only 15 to 17% for a concentrate which would be produced by using a second anion exchange resin as a replacement for hydroxyapatite. It is also noted that the respective ratios of the coagulation factors making up the prothrombic complex are comparable in the concentrates obtained by both aforementioned methods.

1. A method for preparing a prothrombic complex composition comprising the following steps:
a) providing a supernatant of a plasma cryo-precipitate;
b) applying said supernatant on an anion exchange resin, and eluting in an eluate containing said complex and proteins having a high molecular weight
c) applying the eluate resulting from step b) on a hydroxyapatite column; and
d) eluting in an eluent containing said complex.

2. The method according to claim 1 comprising an additional pre-elution step c1), said pre-elution being carried out at a pH comprised between 6.5 and 8.5, with a sodium phosphate or potassium phosphate buffer, in a concentration from 0.005 to 0.05 M, said buffer also comprising NaCl in a concentration of 0.25 M.

3. The method according to claim 1 wherein the elution of step d) is carried out with a potassium phosphate buffer of 0.5 M, 0.075 M NaCl, pH 8.

4. The method according to claim 1 further comprising at least one additional step for viral inactivation of the eluate resulting from at least one of: step b) and of the eluate resulting from step d).

5. The method according to claim 4 wherein said at least one viral inactivation step is carried out on the eluate resulting from step b) in the form of a solvent-detergent treatment, in the presence of a Tween (polysorbate 80)—TnBP mixture, with 1% (v/v) polysorbate 80—0.3% (v/v) TnBP mixture.

6. The method according to claim 4 further comprising at least one additional viral removal step carried out on the eluate resulting from step d) in the form of nanofiltration, on a filter having a porosity from 15 to 100 nm.

7. The method according to claim 1 further comprising at least one additional diafiltration-ultrafiltration step after at least one of: step b) and step d).

8. The method according to claim 1 wherein step b) comprises two sub-steps implemented on two distinct anion exchange resins.

9. The method according to claim 1 wherein the anion exchange resin of step b) has a positively charged group selected from diethylaminoethane (DEAE), polyethyleneimine (PEI) and quaternary aminoethane (QAE), said anion exchange resin being of the DEAE type.

10. The method according to claim 1 further comprising the addition of an inhibitor of thrombin, including antithrombin III or a mixture of antithrombin III and of heparin after step b) or after step d).

11. The method according to claim 1 wherein the composition further comprises other proteins dependent on vitamin K, including at least one of: such as the proteins C, S and Z.

12. The method according to claim 1 further comprising a final additional formulation step, including a least one of: by freeze-drying and adding adjuvants or pharmaceutically acceptable carriers.

13. A prothrombic complex composition obtainable by
a) providing a supernatant of a plasma cryo-precipitate;
b) applying said supernatant on an anion exchange resin, and eluting in an eluate containing said complex and proteins having a high molecular weight;
c) applying the eluate resulting from step b) on a hydroxyapatite column; and
d) eluting in an eluent containing said complex; and at least one of:
for which the concentration of immunoglobulins and of IgM is less than 0.1%, for which the fibrinogen concentration is less than 0.1%, for which the fibronectin concentration is less than 0.1% or for which the concentration of factors of the complement is less than 0.1%.

14. The prothrombic complex composition according to claim 13, for which the average specific activity of the FIX is of at least 4 IU per mg of proteins.

15. The prothrombic complex composition according to claim 13 further comprising protein C, protein S and protein Z.

16. The prothrombic complex composition according to claim 15, wherein the proteins dependant on vitamin K, constituted by the Factor II (FII), the Factor VII (FVII), the Factor IX (FIX), the Factor X (FX), the protein C, the protein S and the protein Z, represent at least 80%, of the total proteins of the composition.

17. The prothrombic complex composition according to claims 13 as a drug.

18. The prothrombic complex composition according to claim 13 as a drug for treating and preventing hemorrhagic accidents related to deficiency in factors dependent on vitamin K, or to overdosage of antivitamin K.

19. The prothrombic complex composition according to claim 13 as a drug for the treatment and prevention of hemorrhagic accidents related to a constitutional or acquired deficiency in Factor II or in Factor X.
